(19) United States
(12) Patent Application Publication
(10) Pub. No.: US 2018/0271444 A1
(43) Pub. Date: Sep. 27, 2018

(54) UNITARY TEMPLATE FOR NEUROLOGICAL MONITORING ELECTRODES

(71) Applicant: Rhythmlink International, LLC, Columbia, SC (US)

(72) Inventor: Harrison M. Floyd, Columbia, SC (US)

(73) Assignee: Rhythmlink International, LLC

(21) Appl. No.: 15/926,047

(22) Filed: Mar. 20, 2018

Related U.S. Application Data
(60) Provisional application No. 62/474,222, filed on Mar. 21, 2017.

(51) Int. Cl. A61B 5/00; A61B 5/0478; A61B 5/04

(52) U.S. Cl. CPC A61B 5/6841; A61B 5/6814; A61B 5/6831; A61B 5/04001; A61B 5/4064; A61B 5/0478

(57) ABSTRACT

A unitary template, that is, a template that is made in one piece with contiguous, integral straps or cords, the ends of which can be joined together to form the completed template is disclosed. The material of which it is made may be inexpensive and accommodates a reasonable degree of flexibility in fitting patients whose heads are of different sizes and additional sizing flexibility by selectably expandable sections. The unitary template may be custom made based a few measurements of the patient's head, and then defined on a sheet of material by printing for hand cutting, by perforations for separating from the balance of the sheet or laser cutting

### TECHNOLOGICAL FIELD

This disclosure relates generally to neurological monitoring and specifically to accurate placement of neurological electrodes.

### BACKGROUND

Neurological monitoring is done as a part of neurological research and during surgical operations. Neurological monitoring involves attaching electrodes to the patient's head and connecting the electrodes electrically to amplifiers for display of the electrical activity of the patient's brain.

The electrodes are positioned in specific parts of the head for several reasons and primarily to provide output that can be compared from one monitoring event to the next. Technicians are trained to know these locations and there are international standards for electrode placement (i.e. the “10-20” system) that are generally followed by those trained in neurological monitoring.

To facilitate correct placement of the electrodes, a template may be placed on the head of the patient. A template serves as a map for locating the standard positions for electrodes. Harnesses are templates that have the electrodes already in place. Templates and harnesses are made of straps that are sewn together in a web-like construction. The positions of the electrodes may be indicated at the intersection of the straps and also between intersections.

Of course, the template or harness, are made with care and precision, and be put on the patient's head correctly and oriented properly for it to effectively guide the positioning of the electrodes, although absolute precision is not required and the heads of humans vary somewhat in size and shape.

A better template would be of advantage particularly in circumstances where technicians are not available to position the electrodes.

### SUMMARY

This disclosure teaches a unitary template, that is, a template that is made in one piece with contiguous, integral straps. The ends of which are joined together, for example with hook and loop fasteners, “key and slot” fastening, or adhesives, to form the completed, three-dimensional template. Moreover, the material of which it is made may be inexpensive and can accommodate a reasonable degree of flexibility in fitting patients whose heads are of different sizes and shapes or be custom-made.

An aspect of the disclosure is a device that is a template for locating electrodes on a patient's head, the template having rows and columns integrally connected to each other, that is, the rows and columns are made of one piece of material. In one example of the present template, the template has one row and five columns. The row crosses the five columns with one of the five columns being the central column and with two columns on its first side and two columns on its second side, opposite the first side of the central column. The central column has a first junction on a first end and a second junction on an opposing, second end. The first ends of the two columns on the first side of the central column and the first ends of the two columns on the second side of the central column are attachable to the first junction of the central column. Similarly, the second ends of the two columns on the first side of the central column and the second ends of the two columns on the second side of the central column are attachable to the second junction of the central column. When rows and columns are connected, the unitary template forms a template conforming to the head of a patient.

In another example, the template has one row and three columns. In other examples, there may be more than one row integral with several columns.

The row and the five columns are marked to indicate the locations of electrodes. For example, the central column may be marked with five locations for electrodes, the first column may be marked with four locations for electrodes, the fourth column may be marked with four locations for electrodes, the second column may be marked with three locations for electrodes, the third column may be marked with three locations for electrodes, and the row may be marked for five locations for electrodes including three locations of the five that coincide with locations on the columns.

Each location may also carry a number or a code to identify the electrode that is to be placed at each location. The row carries a nasion marker or an inion marker or both, one on each of its opposing ends.

A feature of the disclosure is that the rows and columns of the template may be made with a material that has expandable sections, sections that can be expanded by pulling on its ends and which expandable sections may be formed by features such as plural cuts or plural folds that allow expansion, and combinations thereof.

Another feature of the present template is that the template made be custom made for a patient. A few measurements are made of the patient's head and used to program a device that produces a flat template which can be cut, laser cut, perforated or three-dimensionally printed so as to be a flat, unitary version of the present template, perhaps printed with the positions marked.

A feature of the disclosure is that the rows and the columns are made of cords and that the locations on the cords and identities of the electrodes for those locations may be marked by tags.

A feature of the disclosure is that the device may include a bolo, which is an adhesive bridge piece or interlocking loops to connect the first end of the central column with the opposing second end of the central column.

These and other features and their advantages will be apparent to those skilled in the art of neurological monitoring from a careful reading of the detailed descriptions of embodiments accompanied by the drawings.

### BRIEF DESCRIPTION OF THE DRAWINGS

In the figures,

FIG. 1 is a top right perspective view of a template according to the prior art;

FIG. 2 is a plan view of a unitary template before assembly, according to an aspect of the disclosure;

FIG. 3 is a top right perspective view of a template assembled from the unitary template of FIG. 2 with rows and columns connected, according to an aspect of the disclosure;

FIG. 4 is a perspective view of a detail of one way the rows and columns can be connected; according to an aspect of the disclosure;

FIG. 5 is a perspective view of a first example of a selectively expandable section for a unitary template, including an expandable cut pattern, according to an aspect of the disclosure;

FIG. 6 is a perspective view of a second example of a selectively expandable section for a unitary template, including an expandable a cut pattern, according to an aspect of the disclosure;

FIG. 7 is a perspective view of a selectively expandable section for a unitary template formed with an accordion fold of template material, according to an aspect of the disclosure;

FIG. 8 is a flow diagram of a process for manufacturing a custom fitted template; according to an aspect of the disclosure;

FIG. 9 is a perspective view of an apparatus for producing a custom-fitted template, according to the present disclosure;

FIG. 10 is an image of a custom-fitted template attached with perforations to a sheet of template stock, according to an aspect of the disclosure;

FIG. 11 is a right side perspective view of a head of a patient with an assembled unitary template made of cords, the cords having tags attached to show electrode positions, according to an aspect of the disclosure;

FIG. 12 is a left side perspective view of the manikin head of FIG. 11, according to an aspect of the disclosure;

FIG. 13 is a top view of the manikin head of FIGS. 11 and 12, according to an aspect of the disclosure;

FIG. 14 shows a portion of a template with a chin strap to secure the two ends of the row together, according to an aspect of the disclosure;

FIG. 15 shows a portion of a template with two side straps secured together by a loop, according to an aspect of the disclosure; and

FIG. 16 shows a portion of a template with two side straps together with a bolo slide, according to an aspect of the disclosure.

### DETAILED DESCRIPTION

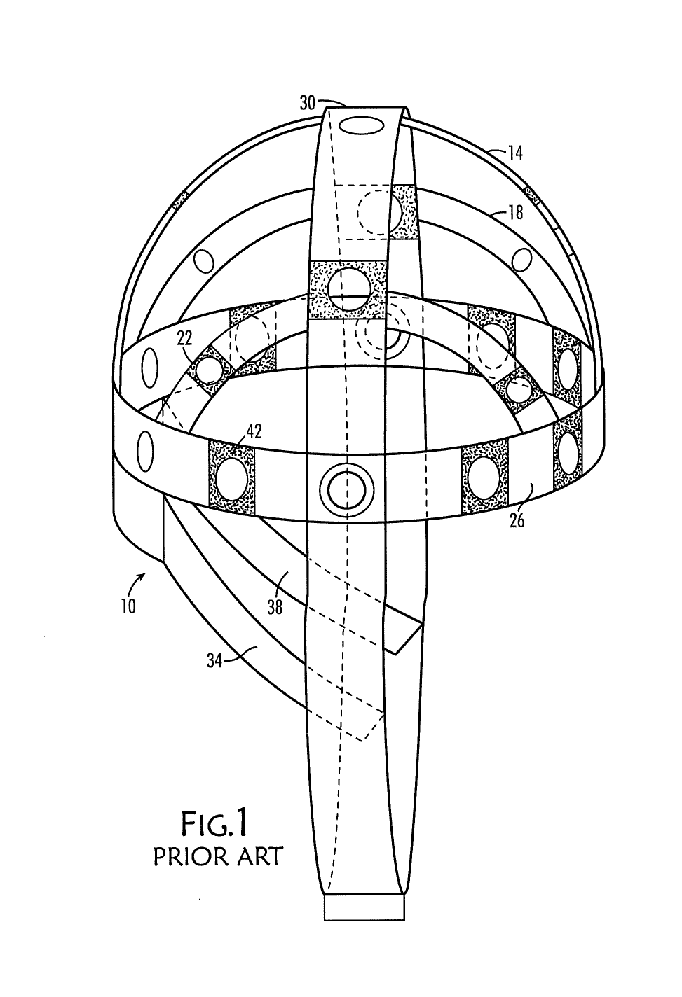

The disclosed device is a unitary template, which is a template made of a piece of material in contrast to a prior art templates such as that shown in FIG. 1, which is made of strips of material sewn together. Unitary, integral, and single piece are used herein to mean that the template is not made of joined sections and is physically formed by subtracting mass from a starting material.

Turning now to FIG. 1, a template 10 made according to the prior art is shown is made of streaps 14, 18, 22, 26, 30, 34, and 38 that are separately made and are be sewn together in the correct positions to form template 10. At intervals along straps 14, 18, 22, 26, 30 34, and 38, locations 42 are provided for electrodes (not shown) to be attached. Straps 14, 18, 22, 26, 30, 34, and 38 may be made of an elastic or stretchable woven fabric to accommodate patients whose heads vary in size and shape.

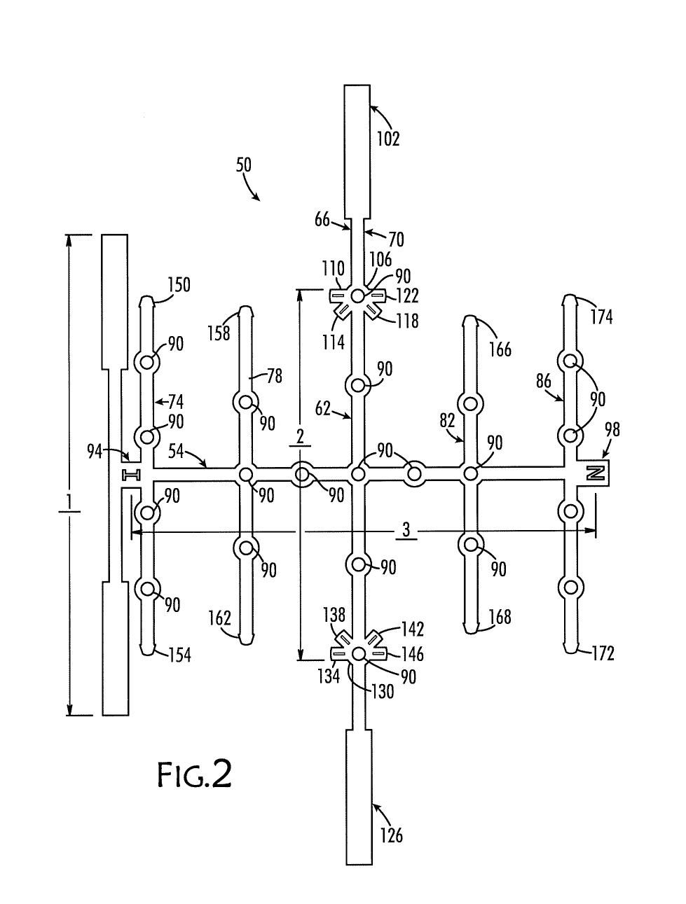

FIG. 2 illustrates template 50, which is made of a single piece of material in a shape that fits on a human head. Template 50 has a row 54 integrally and contiguously connected with and bisecting columns 58, 62, 74, 78, 82, 86, which are collectively made of one piece and not fastened together from separate pieces. Construction is faster, cheaper, and the relationship among electrode positions more precise than in the case of a template 10. Four columns 74, 78, 82, 86, are deployed to the sides of a central column 62. Central column 62 has a first side 66 and an opposing second side 70. To the left of first side 66 of central column 62 is a first column 74 and a second column 78. To the right of second side 70 of central column 62 is a third column 82 and a fourth column 86. Template 50 has, in this example, 21 electrode locations 90 on central column 62, first column 74, second column 78, third column 82 and fourth column 86 and also row 54. Electrode locations 90 are spaced apart and positioned so that, when template 50 is connected as described below and electrodes (not shown) are attached at the nineteen electrode locations 90, and with template 50 in place on the head of a patient, the electrode locations are then in the approximate positions on the head of the patient for the electrodes attached at the electrode locations 90 for neurological monitoring.

First column 74 and fourth column 86 are longer than second column 78 and third column 82. First column 74 and fourth column 86 have four electrode locations 90 spaced at intervals, with two electrode locations 90 on either side of row 54. Both second column 78 and third column 82 have three electrode locations 90 spaced at intervals thereon, including one electrode location 90 on second column 78 and third column 82 where they cross row 54. Central column 62 has five electrode locations 90 spaced at intervals with one of those five electrode locations 90 on row 54. Thus, there are five electrode locations 90 on row 54 of which one location of the five electrode locations 90 coincides with second column 78, another electrode location 90 coincides with central column 58, and a third electrode location coincides with third column 82. Two more electrode locations are on row 54, one between central column 62 and second column 78 and the other between central column 62 and third column 83. Altogether, there are 21 electrode locations 90 on template 50 as shown in FIG. 2.

Row 54 has a first end 94 and a second end 98. First end 94 is marked with a letter I to indicate the Inion; second end 98 is marked with a letter N to indicate Nasion. The portion of first end 94 with the letter I is to be used to position template 50 on the patient so the location of the letter I corresponds to the inion or bump on the back of the patient's head. The portion of second end 98 if row 54 with the letter N is to be used to position template 50 on the patient so that the location of the letter N is on the bridge of the patient's nose.

Central column 62 has two junctions integrally connected to the ends of first column 74, second column 78, a third column 82 and fourth column 86. Specifically, on a first end 102 of central column 62, a junction 106 has first connection 110, a second connection 114, a third connection 118, and a fourth connection 122. On a second end 126 of central column 62, there is a junction 130 that has a fifth connection 134, a sixth connection 138, a seventh connection 142, and an eighth connection 146. The second ends 154, 162, 168, and 172 of first column 74, second column 78, third column 82, and fourth column 86, respectively, are attached to fifth connection 134, sixth connection 138, seventh connection 142, and eighth connection 146 of second junction 126.

Specifically, first column 74 has a first end 150 and an opposing second end 154 and second column 78 has a first end 158 and an opposing second end 162. Third column 82 has a first end 166 and an opposing second end 170; fourth column has a first end 174 and an opposing second end 178. First end 150 of first column 74, first end 158 of second column 78, first end 166 of third column 82 and first end 174 of fourth column 86 are attached to first connection 110, second connection 114, third connection 118, and fourth connection 122, respectively, of junction 106. Second end 154 of first column 74, second end 162 of second column 78, second end 168 of third column 82 and second end 172 of fourth column are attached to second junction 126

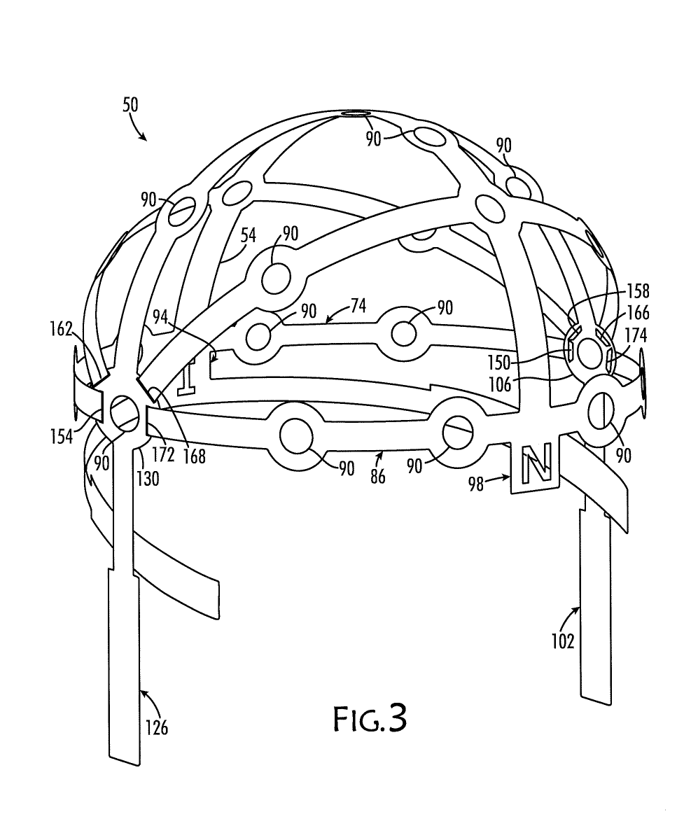

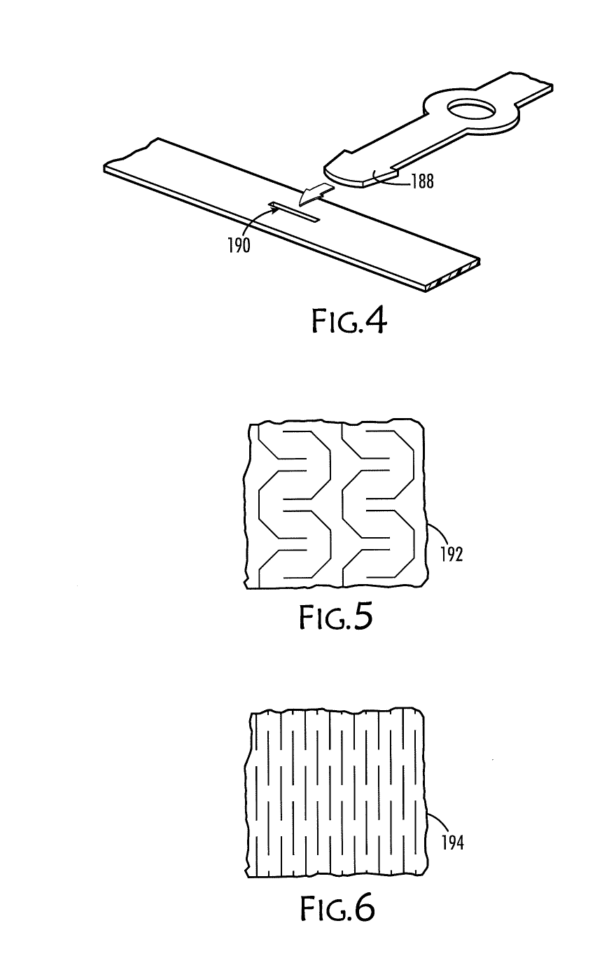

The mode of attachment is not critical. For example, adhesives on first junction 106 and on second junction 126 (or on the ends of columns 74, 78, 82, 86) with release paper covers may be used. An alternative attachment mode is shown in FIG. 4 where slots 200 receive a key 188, which is a shaped end of a column such as first end 150 of first column 74 being received in junction first 106. The shape of key 188 is such that key 188 slips into slot 190 by bending it slightly on insertion, and resists removal.

The result of connecting column ends to junctions as described above is shown in FIG. 3. FIG. 3 is a right top perspective view.

Similar templates may be made of different numbers of row and columns. For example, a template can be made with at least one row and at least two columns. Templates may be made with two rows and three columns, for example, for a ten-electrode template. The numbers of rows and columns may be chosen once the basic electrode pattern is established, and which may be custom pattern for electrodes or a standard 10-20 pattern.

Template 50 may be made by removing material from a large solid piece for stacking, storage and sihpping, and then by attaching the ends to conform the template to the shape of a head at the time of use. The template may be alternatively be formed by three-dimensional printing either as a two dimensional pattern with thickness or a three-dimensional pattern with thickness.

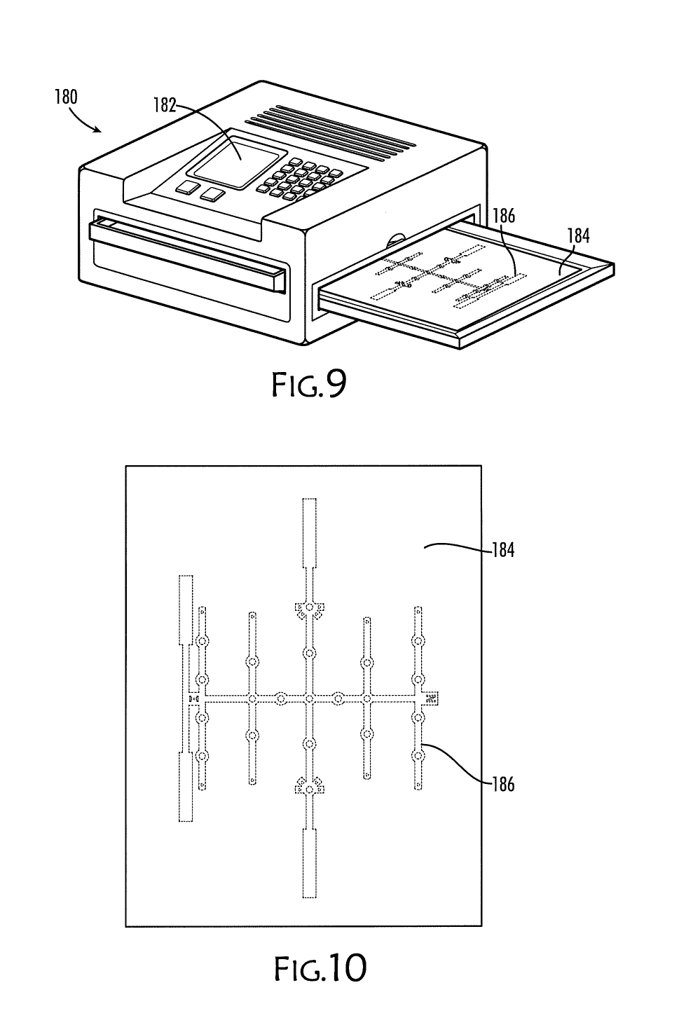

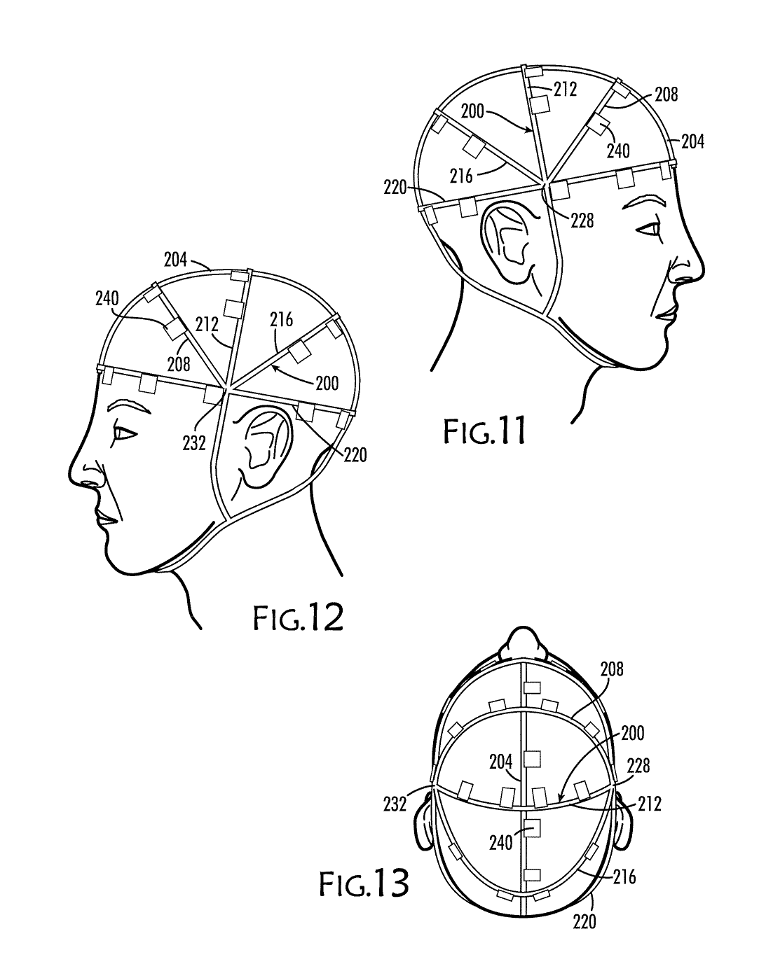

Template 50 may be made from a flat sheet to fit the head of a specific patient at the time of need. A child may require a template 50 that is smaller, for example. Simple measurements, as shown in FIG. 2 and labeled 1, 2, and 3 may be taken and the overall flat template shaped scaled to meet those measurements. FIG. 8 illustrates a flow chart for this process. The scaled template may then be printed or cut from a piece of material that is sufficiently rigid and strong and flexible to permit it to be cut or perforated to show how template 50 may be separated from surrounding material, or laser cut. A machine 180, as illustrated in FIG. 9, may be adapted to receive measurements via a user interface 182 and print or perforate a flat sheet 184, shown in FIG. 10, containing a template 50 scaled for the user and that may be separated from flat sheet 184. A laser cutter or die cutter are suitable alternatives for cutting template 50 from flat sheets 184 cord.

Template 50 can be made of prior art materials that have resilience:

woven materials with resilience. In the alternative, unitary templates 50 may be made with a material that is lower cost, strong and resilient, such as synthetic, non-woven, laminated, fibrous material. An example of such material is commonly used for house wrap, and sold by E. I. DuPont de Nemours & Company, Inc., under the trademark TYVEK.

As an alternative to material that has resilience, template 50 may be made with selectively expandable portions. In row 54 of template 50, for example, which crosses the “crown” of template 50 when assembled and worn a selectively expandable portion may be useful to accommodate the shape of the crown. FIGS. 5 and 6 show two ways of using plural cuts in the material of which template 50 is made to provide “give” or additional length when the cut portions open up under lateral tension. FIG. 6 shows an “expanded metal” cut 194 in a diagram of a physical sample. FIG. 5 shows a more intricate, nested cut 196, which provides more stretch per unit length of material with perhaps forming a row or two of plural cuts.

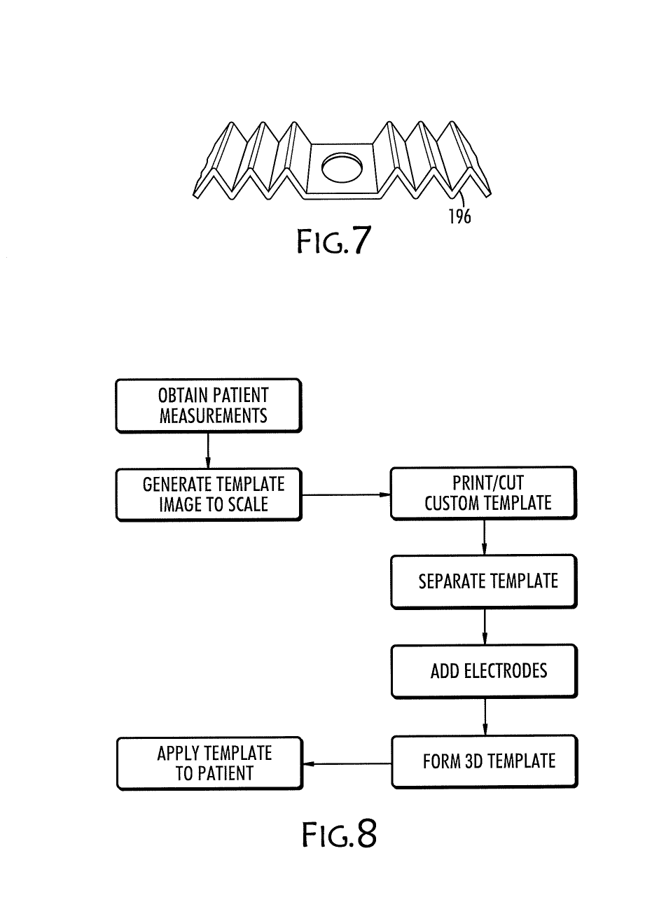

FIG. 7 shows a portion of a template 50 formed with an “accordion” fold 198 that naturally opens up and tends to flatten as tension is applied. Combinations of plural cuts and accordion folds and materials also may be used to accommodate variations in head size and shape.11, 12, and 13 illustrate an alternative template 200. Like template 50, template 200 is made of a piece of material. Unlike template 50, unitary template 200 is made of cords that are fastened together in the same manner as described above. Template 200 has a row cord 204, a central cord 212, a first column cord 212, a second column cord 216, a third column cord 220, and a fourth column cord 224. Row cord 204, first column cord 208, second column cord 212, third column cord 216, and fourth column cord 220 are attached to a first joint 228 and to a second joint 232 to form template 200 that fits on the head of a patient 236. Tags 240 can be attached, perhaps by crimping, to row cord 204, first column cord 208, second column cord 212, third column cord 216, fourth column cord 220, fifth column cord 224, to identify the locations for, and numbers and codes of, the electrodes.

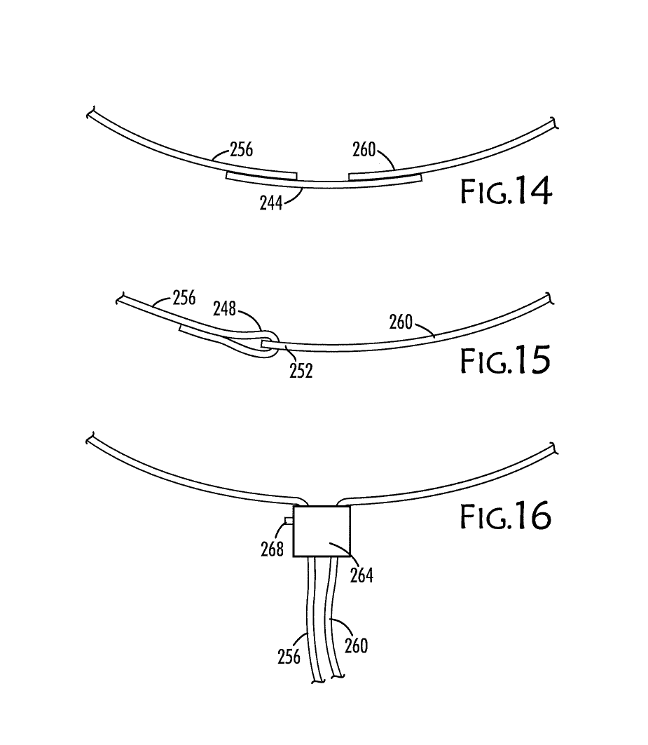

First end 102 and second end 126 of central column 62 need to be joined when template 50 or template 200 is worn by a patient. FIGS. 14, 15, and 16 show three alternative ways to fasten first end 102 to second end 126. A bridge piece 244 may be used that adheres to first end 102 and to second end 126, as illustrated in FIG. 14. Most suitably for unitary template 200, FIG. 15 illustrates interlocking loops 248, 252 in a first end 256 and a second end 260 of cord 212. However, a third example is a bolo-type fastener 264 with a spring button 268, as shown in FIG. 16.

Many modifications and substitutions may be made in the foregoing detailed description without departing from the spirit and scope of the disclosure which is defined in the appended claims.

What is claimed is:

1. A device for use in neurological monitoring of a patient's brain, said device comprising a template having electrode positions designated thereon, said template being a flat sheet and including a row and at least two columns integrally connected with said row, wherein a first column of said at least two columns has a first end and a second end, a second column of said at least two columns has a first end and a second end, said first end of said first column being attachable to said first end of said second column, said first end of said second column being attachable to said second end of said second column, said row being dimensioned to run from the nose of the patient to the back of the head of the patient, said at least two columns being spaced apart and extending laterally with respect to said row, wherein when said first end of said first column is attached to said first end of said second column and said second end of said first column is attached to said second end of said second column, said flat sheet curves to define a template conforming to the patient, and wherein said row and said at least two columns carry electrode positions.

2. The device of claim 1, wherein said at least two columns is three columns, including a central column with a first side and an second side, and wherein a first column is on said first side of said central column and a second column is on a second side of said central column, and wherein said central column has a first junction and a second junction, and wherein said first column has a first end and a second end and said second column has a first end and a second end, and wherein said first end of said first column and said first end of said second column are attached to said first junction of said central column and said second end of said first column and said second end of said second column at attached to said second junction of said central column.

3. The device of claim 2, wherein said first junction of said central column and said second junction of central column have slots and wherein said first end and said second end of said first column and said first end and said second end of said second column have expands that fit into said slots of said first junction of said central column and said second junction of said central column.

4. The device of claim 2, wherein said at least two columns is five columns.

5. The device of claim 2, wherein said row has a first end and a second end, said first end carrying a nasion marker and said second end carrying an inion marker.

6. The device of claim 2, wherein an electrode position of said electrode positions carries a code designating said electrode position.

7. The device of claim 2, wherein said electrode locations are marked by tags.

8. The device of claim 2, wherein said row is made with an expandable section.

9. The device of claim 8, wherein said expandable section is formed of plural cuts in said row.

10. The device of claim 8, wherein said expandable section is formed of accordion folds.

11. The device of claim 2, wherein said template is die cut from said flat sheet.

12. The device of claim 2, wherein said template is produced on a perforated sheet.

13. The device of claim 2, wherein said template is scaled for a patient.
